(19) United States
(12) Patent Application Publication
(10) Pub. No.: US 2007/0224274 A1
(43) Pub. Date: Sep. 27, 2007

(54) Polymer Particles Containing Active Agents

(76) Inventor: Werner Siol, Darmstadt (DE)

Correspondence Address: HUTCHISON LAW GROUP PLLC, PO BOX 31686, RALEIGH, NC, 27612, US

(21) Appl. No.: 10/594,365

(22) PCT Filed: Apr. 2, 2005
(86) PCT No.: PCT/EP05/03493

§ 371 Date: Sep. 27, 2006

(30) Foreign Application Priority Data
Apr. 5, 2004 (DE) 102004016685.4

(51) Int. Cl. A61K 47/32; A61K 47/48; C08F 220/12

(52) U.S. Cl. 424/489

(57) ABSTRACT

The invention relates to active ingredient-containing polymer particles comprising a vinyl polymer from 10-80% by weight monomers comprising amino and/or carboxyl groups, which is insoluble in one part of the pH range 0-10 and is soluble in another part of the range, wherein these active ingredient-containing polymer particles comprise 3-1000 parts of active ingredient per part of vinyl polymer, have a particle size in the range 20 nm-8 μm and the vinyl polymer is >50% by weight composed of polymers having a molecular weight of <100 000 daltons.

### FIELD OF THE INVENTION

The invention relates to active ingredient-containing polymer particles in the size range from 20 nm to 8 μm which are insoluble in water in part of the pH range 0-10 and are soluble in another part of the range.

### PRIOR ART

The synthesis of plastics particles by emulsion polymerization has developed to a higher level. Thus, polymer particles with activated surfaces are employed in immunodiagnosis (e.g. DE 31 16 995). Particularly uniform particles 10 μm in size as calibration standards were the first product manufactured commercially in space (see Vanderhoff et al. U.S. Pat. No. 5,106,903); Ugelstad describes low molecular weight plastics particles of, for example, PVC or polystyrene which are able to absorb 500 times their own volume of swelling agents (DE 2751867). These particles can also be swollen with active ingredients, e.g. crop protection agents. Vaccines based on styrene/acrylate particles are described in U.S. Pat. No. 4,225,581. It is even possible with specific nanoparticles to overcome the blood-brain barrier (U.S. Pat. No. 6,117,454).

A disadvantage of these active ingredient particles is the circumstance that the particle carriers cannot be absorbed. An exception is reported in a recent patent application (P 103 53 989.1, not yet published). It is reported therein that plastics particles in the 0.01-μm size range which comprise specific oligoesters of glycolic acid and lactic acid with (meth)acrylate end groups satisfy the requirements of bioabsorbable active ingredient carriers. Particles of this type are initially insoluble in water. However, after hydrolysis for example of the lactic ester groups, these particles decompose into water-soluble constituents.

There has been special interest in emulsion polymers as coating agents for medicaments (DE 2135073). Depending on whether these emulsion polymers comprise carboxyl groups or amino groups it is possible therewith to achieve tablet coating materials resistant to gastric juice, soluble in intestinal juice or soluble in gastric juice.

The emulsion polymers are in these cases employed directly or as redispersible powders (DE 3208791).

Functional acrylate dispersions of this type are now also used as a dermal therapeutic system (DE 4310012). Microparticles made by coagulating polymer dispersion and active ingredient are also described (DE 4328069).

### PROBLEM AND SOLUTION

Whereas amino group-containing (i.e. soluble in gastric juice) or carboxyl group-containing (i.e. soluble in intestinal juice) emulsion polymers are widely used as tablet-coating film (i.e. as particle composite), these emulsion polymers are not generally employed as aqueous dispersion, i.e. as single particles, as active ingredient carrier.

Preference is given here to liposomes as medicament carriers.

There is still no simple technical solution to the targeted release of liquid active ingredients or dispersions.

It has now been found that active ingredient-containing polymer particles in a size range from 20 nm to 8 μm, comprising from 3 to 1000 parts of active ingredient and 1 part of vinyl polymer composed of 10-80% by weight monomers comprising amino and/or carboxyl groups, which is insoluble in part of the pH range 0-10 and is soluble in another part of the range, and which consists to the extent of >50% by weight of polymers having a molecular weight of <100 000 daltons, are most suitable as particulate active ingredient carriers. Vinyl polymers having a molecular weight of <20 000 or <5000 are particularly preferred in this connection.

Specifically of industrial interest are those active ingredient-containing polymer particles which are >60% by weight or entirely composed of the abovementioned vinyl polymers and active ingredients.

Particularly preferred active ingredient-containing polymer particles are those whose polymer component consists of
A) 20-90% by weight alkyl esters of acrylic and/or methacrylic acid,
B) 80-10% by weight monomers having carboxyl groups and/or amino groups,
C) 0-40% by weight further monomers copolymerizable with A) and B).

Alkyl esters which should be mentioned as A) are esters having 1-8 C atoms in the alkyl radical, in particular methyl and ethyl acrylate and methacrylate. Suitable acid monomers B) are acrylic acid and, in particular, methacrylic acid. Further acid monomers are maleic, fumaric and itaconic acid and monoesters of these acids.

Suitable monomers B) having amino groups are, for example, vinylimidazole, monoalkylamino- and dialkylaminoalkyl esters or monoalkylamino- or dialkylaminoalkylamides of polymerizable carboxylic acids, e.g. dimethylaminoethyl methacrylate.

Suitable monomers C) are very generally vinyl monomers, e.g. hydroxyethyl methacrylate or styrene.

The polymer particles will generally comprise either only basic or only acidic monomers B).

The ratio of the amounts of monomers A), B) and C) depends on the requirements of the release of active ingredients.

Active ingredient-containing particles of particular interest are those whose polymer components consist only of monomers A) and B).

Examples of interest are polymer components composed of 50% by weight ethyl acrylate and 50% by weight methacrylic acid. Particularly interesting active ingredient-containing polymer particles are those whose polymer component consists only of methacrylate monomers, e.g. of 40-80% by weight methyl methacrylate and 60-20% by weight methacrylic acid.

It may be pointed out even at this juncture that the solubility behavior of the polymer particles loaded with active ingredient depends very substantially on the hydrophobicity of the active ingredient and its functional groups. This emerges simply from the active ingredient/polymer particle ratio of amounts.

These active ingredient-containing polymer particles generally consist of 1 part of pH-sensitive polymer and 3-1000 parts of active ingredient. This means that the polymer particles employed here can absorb up to 1000 times their own weight of active ingredient. A particularly interesting ratio by weight of polymer particles to active ingredient is in the range from 1:3 to 1:500, with preference for the range from 1:5 to 1:300 and in particular from 1:10 to 1:200.

In the case of liquid active ingredients, the active ingredient represents the solvent or plasticizer for the polymer particles. In certain cases, this leads to the active ingredient-containing particles dissolving faster in water than the polymer particles without active ingredient when the pH changes.

Procedure for the Invention.

The polymer particles are generally synthesized by emulsion polymerization by the feed method as described for example in DE 2135073.

In this case, the size of the particles is most simply controlled by the amount of emulsifier initially present.

Polymer particles in the range 20 nm-500 nm can be obtained in this way. Larger particles can be attained by the seed latex method (see example 2). Emulsion polymers containing carboxyl groups are usually prepared with anionic emulsifiers such as, for example, sodium lauryl sulfate, and polymers containing amino groups with cationic or nonionic surfactants such as, for example, ethoxylated fatty alcohols. The polymerization is generally carried out under an inert gas, e.g. nitrogen. The initiators employed are the systems used for emulsion polymerizations, such as ammonium peroxodisulfate or the sodium salt of 4,4′-dicyano-4,4′-azovaleric acid. If it is wished to control the molecular weight of the polymers by the amount of initiator employed, it is also possible to use org. peroxides such as, for example, t-butyl perpivalate in the feed.

However, the molecular weight of the polymers is normally adjusted with the aid of polymerization regulators such as mercaptans. Mention should be made in this connection of alkanethiols and in particular of esters of thioglycolic acid or mercaptopropionic acid, e.g. 2-ethylhexyl thioglycolate, in proportions of 0.1-10% by weight, preferably 0.3-5% by weight, based on the solid polymer.

Especially when larger proportions of mercaptans are employed, deodorization by degassing under reduced pressure is advisable after completion of the polymerization. Controlling the molecular weight of the polymers is important for the uptake of large amounts of active ingredient. For this reason, at least one part of the polymer (e.g. at least 50% by weight or better >90% by weight should have a molecular weight of <100 000 daltons. It is better if the molecular weight (Mw) is

<30 000 or preferably <20 000 daltons. It is particularly advantageous for the molecular weight to be in the range from 1000 to 10 000 daltons and particularly preferably in the range from 2000 to 8000 daltons.

It is of interest that these short-chain polymers form in combination with the active ingredients, very stable particles which, for example as particles containing carboxyl groups, are very stable in the acidic range, e.g. pH 2-3, whereas they disintegrate in the neutral to alkaline range within seconds or fractions of seconds (depending on the size and active ingredient content of the particles).

In some circumstances, the great stability of the active ingredient-containing polymer particles in the insoluble pH range is a question of osmosis which actually forces the active ingredients as a type of solvent or diluent into the particles.

Once formed, the active ingredient particles, e.g. as aqueous dispersions, are stable at a given pH for months. If the particles undergo sedimentation or creaming, because of their size and a density differing from the aqueous phase, these active ingredient-containing polymer particles can be redispersed by brief shaking.

There are scarcely any limits on the choice of active ingredients. Active ingredients mean in the widest sense medicinal substances, cosmetic active ingredients, UV stabilizers, perfume oils, veterinary medicaments, and very generally substances displaying a physiological effect. Particularly suitable active ingredients are liquid or oily substances with low solubility in water. Active ingredients having a solubility of <50 g/l or preferably <10 g/l of water deserve particular interest. Solids can be incorporated at elevated temperature or in the presence of solvents, e.g. butyl acetate.

Ordinarily, the solvent is removed again after the active ingredient has been incorporated into the polymer particles (e.g. distilled out).

Care must be taken, especially with active ingredients having amino or acidic groups, that the active ingredient does not bring the polymer particles into a pH range in which they dissolve. High-melting substances with low solubility in organic solvents are suitable only with restrictions as active ingredients. The particles must be stabilized if appropriate by metering in an emulsifier. However, in general, addition of emulsifier is unnecessary or necessary only for incorporation of the active ingredients.

When the active ingredient/polymer ratios are very high, it may be advantageous to buffer the aqueous phase by small amounts of buffer, e.g. in the ppm range, in the pH range insoluble for the particles, e.g. in the pH range 3-4 for polymer particles containing carboxyl groups. However, ordinarily, no buffer will be employed.

The active ingredients are generally incorporated into the polymer particles in aqueous dispersion.

At elevated temperature, the incorporation preferably takes place in stirring apparatuses.

Incorporation of liquids at room temperature can take place by simple shaking or preferably in overhead mixers. Stirring will generally be only slow. Application of large shear forces (turbomixers etc) must be avoided.

It may be pointed out once again that the pH-sensitive polymer/active ingredient combinations of the invention are very stable. Thus, the active ingredient is thoroughly absorbed by the polymer particles to a certain extent as osmotic diluent.

It is for this reason also possible to attain very fine active ingredient-containing polymer particles (diameter e.g. 50 nm) which are otherwise not obtainable by simple fragmentation even with high shear forces.

The active ingredient-containing polymer particles will ordinarily be employed directly as aqueous dispersions. However, it is also possible, especially with solid active ingredients, for example to freeze dry the active ingredient-containing polymer dispersions in order thus to obtain the fine active ingredient-containing polymer particles as solid, which can then be used in a wide variety of formulations for particularly rapid release of active ingredients. The active ingredient-containing polymer particles are, however, ordinarily employed with 0.25-999 parts of water per part of active ingredient-containing polymer particles, i.e. as aqueous dispersion with a water content in the range 20-99.9% by weight, preferably in the range 40-95% by weight. If appropriate, preservatives such as, for example, ethyl p-hydroxybenzoate are added. Although it is easily possible for an active ingredient-containing polymer dispersion which has undergone sedimentation or creaming if appropriate to be rehomogenized by shaking, it is also possible to take measures from the outset to prevent sedimentation or creaming of the particles, e.g. by adjusting the density of the active ingredient-containing polymer particles to the density of the aqueous phase or else by increasing the viscosity of the aqueous phase by water-soluble thickeners.

The size of the active ingredient-containing polymer particles is primarily determined by the size of the polymer particles and the active ingredient/polymer ratio. Thus, the mass of a polymer particle 100 nm in size which takes up 7 times the amount of active ingredient will increase 8-fold. This means if the active ingredient and polymer particles are of comparable density that the diameter doubles to 200 nm. Correspondingly, a particle (diameter 100 nm) which takes up 124 times the amount of active ingredient, corresponding to a 125-fold increase in the total mass, will be enlarged to a diameter of 500 nm (see example 2).

As stated, the pH-sensitive, controlled polymer particles may take up as much as 1000 times their own volume of active ingredient, corresponding to a 10-fold increase in the particle size.

Thus, in principle, active ingredient-containing polymer particles in the range 0.02-20 μm can be obtained, the size range 0.04-12 μm being preferred and the range 0.05-8 μm being particularly preferred. Particularly favorable active ingredient/polymer ratios can be achieved with active ingredient-containing polymer particles in the range >2-<8 μm. The active ingredient-containing polymer particles are normally spherical with a smooth surface. They are preferably non-coagulated, freely movable single particles in which the active ingredient is homogeneously dispersed. The particles are preferably monodisperse, i.e. >80% by weight of all the particles show the same particle diameter. It is moreover possible for active ingredient-containing polymer particles with a bimodal or multimodal particle size distribution to be employed. This is of interest firstly if it is wished to use the particles in aqueous dispersion with a minimum water content, and secondly this option is appropriate if it is wished to obtain a dispersion of different active ingredient-containing polymer particles for example with different release conditions.

With these active ingredient-containing polymer particles there is generally rapid release of the active ingredient when the appropriate pH at which the particle is soluble is reached. Slower release of the active ingredients with progressive hydrolysis of crosslinking or hydrophobic groups as described in the unpublished patent application P 10353989.1 is not according to the invention.

This means that the active ingredient-containing polymer particles of the invention are also characterized in that the polymer particles have a content of <1% by weight of monomers of the general formula
(CH₂═CR₁—CO—(—O—CHR₂—CO—)ₘ—O—)ₙ—R₃ (I)
where R₁ and R₂ are independently of one another H or CH₃,
m is 1-20, and
R₃ is an optionally substituted alkyl radical having 1-18 carbon atoms for n=1. or is an optionally substituted alkylidene radical having 2-18 carbon atoms for n=2.

Very particularly preferred active ingredient-containing polymer particles are those whose polymer component comprises none of the abovementioned monomers (I).

Particular advantages of the novel active ingredient-containing polymer particles

It is particularly important for broad application of the active ingredient-containing polymer particles that the chemical composition of the polymers from which these active ingredient particles are constructed has been known for decades as tablet coating material in pharmaceutical formulations. Thus, the route followed by these polymers in the body has been thoroughly investigated. The polymers used in this case differ from the products employed as tablet coating material essentially in the molecular weight. Shorter polymers are employed in the active ingredient-containing plastics particles. Even better degradation can be expected from experience for these shorter chains. An additional point is that, because the polymer particles are able to take up large amounts of active ingredient, the proportion of polymer relative to the active ingredient is very low, e.g. <1% by weight (see example 2).

It is also of interest that the active ingredient-containing plastics particles are very stable in their initial pH range, e.g. pH 3 for polymer particles containing carboxyl groups, whereas they release the active ingredient virtually instantaneously when the pH is changed to 7.

It is possible in principle for 2 different active ingredient-containing polymer particles with 2 active ingredients which have an interfering influence to be used in the same aqueous dispersion if these active ingredients are insoluble in water, and the dispersions comprise only small amounts of emulsifier. It is, of course, necessary for the two types of active ingredient-containing polymer particles to be prepared separately.

It is also significant that the active ingredient-containing polymer particles can also be prepared with very sensitive active ingredients because incorporation of the active ingredients into the polymer particles ordinarily takes place by simple shaking at room temperature.

A circumstance which should be particularly emphasized is the fact that these active ingredient-containing polymer particles are most suitable for administering liquids and oils. On the one hand, it is possible in this way to adjust the amount of the active ingredient by the specified dilution of the dispersion to a specified amount which can be easily measured (10 drops, 1 measuring spoonful etc) and, on the other hand, simple, alcohol-free administration of liquid active ingredients and essences which are insoluble in water is possible for the first time.

The invention also includes a method which comprises swelling 1 part of polymer of an aqueous polymer dispersion of a vinyl polymer which is composed of 10-80% by weight monomers comprising amino and/or carboxyl groups and which is insoluble in one part of the pH range 0-10 and is soluble in another part of the range and which is >50% by weight composed of polymers having a molecular weight of <100 000 daltons, with 3-1000 parts of active ingredient, with formation of active ingredient-containing polymer particles in a size range of 20 nm-8 μm, and administering these active ingredient-containing polymer particles.

The following examples are intended to illustrate the invention but do not represent a restriction.

Examples D1-D3 describe the synthesis of the pH-sensitive polymer dispersions for example polymers containing carboxyl groups,

Examples 1-4 describe the preparation of the active ingredient-containing polymer particles, and

Example 5 describes the release of the active ingredient from these particles.

### EXAMPLE D1 SYNTHESIS OF A FINE-PARTICLE POLYMER DISPERSION CONTAINING CARBOXYL GROUPS WITH UNIFORM PARTICLE SIZE

0.1 g of sodium lauryl sulfate is introduced into 500 g of water in a stirred reactor. Addition of 50 g of a 1% strength solution of potassium peroxodisulfate in water is followed by the metering in, at 80° C., of a mixture of
105 g of methyl methacrylate,
105 g of methacrylic acid,
   0.6 g of 2-ethylhexyl thioglycolate,
   0.2 g of sodium lauryl sulfate,
   2 g of water
      at 80° C. under argon over the course of 3 h. The mixture is then stirred at 80° C. for a further h. A fine-particle dispersion is obtained after filtration through a fine-mesh screen fabric. Solids content: 27%, pH 3, particle diameter 0.15 μm.

### EXAMPLE D2 SYNTHESIS OF A HIGHLY SWELLABLE POLYMER DISPERSION CONTAINING CARBOXYL GROUPS BY THE SEED LATEX METHOD

20 g of dispersion D1 are introduced into 600 g of a 1% strength solution of potassium peroxodisulfate in water in an apparatus as in example D1. At 75° C., a mixture of 85 g of methyl methacrylate, 85 g of methacrylic acid, 7 g of butanethiol, 0.2 g of sodium lauryl sulfate and 3.7 g of water are metered into this dispersion.

After the feeding in is complete, a solution, warmed to 40° C., of 0.14 g of potassium peroxodisulfate and 0.12 g of sodium lauryl sulfate in 40 g of water is metered in. The mixture is then stirred at 80° C. for 1 h.

A dispersion with a solids content of 21%, pH 3, particle size about 0.5 μm is obtained after filtration. The polymer particles are very uniform; they sediment but can easily be shaken up again. The molecular weight of >90% of the polymers is <10 000 daltons. Solution test with change of pH:

on dropwise addition of dispersion D2 to a phosphate buffer solution of pH 7.0 the particles dissolve completely within 30 s.

### EXAMPLE D3 SYNTHESIS OF A FINE-PARTICLE, HIGHLY SWELLABLE POLYMER DISPERSION CONTAINING CARBOXYL GROUPS

In accordance with the method of example D1, a solution of 0.24 g of potassium peroxodisulfate and 0.11 g of sodium lauryl sulfate in 525 g of water is prepared. 3 g of a mixture of 74 g of methyl methacrylate, 73 g of methacrylic acid, 0.28 g of 2-ethylhexyl thioglycolate, 0.2 g of sodium lauryl sulfate and 2 g of water is metered into this at 80° C. Then 6.5 g of butanethiol are added to the remainder of the mixture, and this mixture is metered into the solution over the course of 2 h.

Finally, the mixture is deodorized under reduced pressure (p=500 mbar).

A fine-particle dispersion, solids: 23.4%, pH 3, is obtained,

particle size about 0.1 μm.

On dropwise addition to phosphate buffer of pH 7.0, the particles dissolve in 1 s.

Incorporation of 2-phenylpropanol (1) as model active ingredient

### EXAMPLE 1 ACTIVE INGREDIENT/POLYMER RATIO=14.3/1

2.0 g of dispersion D2 (comprising 0.42 g of polymer), 0.1 g of sodium lauryl sulfate, 40 g of water and 6.0 g of 2. phenylpropanol (1) in a 50 ml laboratory bottle are subjected to cartwheel rotation for 4 h. A stable dispersion with a particle size of about 1.2 μm is obtained.

### EXAMPLE 2 ACTIVE INGREDIENT/POLYMER RATIO=128.6/1

1.0 g of the active ingredient-containing dispersion of example 1, comprising 0.125 g of 2-phenylpropanol (1) and 0.00875 g of polymer, is subjected to cartwheel rotation with 10 g of water and 1.0 g of 2-phenylpropanol (1) for 4 h. A stable dispersion with a particle size of 2.5 μm (uniform) is obtained.

The dispersion has sedimented after standing at RT for 14 days. A homogeneous dispersion is obtained again by brief shaking.

### EXAMPLE 3 ACTIVE INGREDIENT/POLYMER RATIO=40.8/1

0.109 g of dispersion D3 is subjected to cartwheel rotation with 5 mg of sodium lauryl sulfate in 7.8 g of water and 1.016 g of 2-phenylpropanol (1) for 2 h. A stable, fine-particle active ingredient-containing polymer dispersion is obtained.

### EXAMPLE 4 ACTIVE INGREDIENT/POLYMER RATIO=3/1

1.2 g of dispersion D1 are subjected to cartwheel rotation with 10 mg of sodium lauryl sulfate, 7.0 g of water and 1.0 g of 2-phenylpropanol (1). A stable, fine-particle dispersion is obtained.

### EXAMPLE 5 RELEASE OF THE ACTIVE INGREDIENT BY CHANGING THE PH

The active ingredient-containing polymer particles 1.2 μm in size from example 1 are added dropwise to a phosphate buffer solution of pH 7.0. The particles dissolve within 1 s. The test is repeated with the active ingredient-containing polymer particles 2.5 μm in size from example 2. These particles also dissolve at pH 7.0 within 1 s.

1. Active ingredient-containing polymer particles comprising a vinyl polymer from 10-80% by weight monomers comprising amino and/or carboxyl groups, which is the vinyl polymer being insoluble in one part of the pH range 0-10 and is soluble in another part of the range, wherein these the active ingredient-containing polymer particles comprise
3-1000 parts of active ingredient per part of vinyl polymer,
a particle size in the range 20 nm-8 μm and
the vinyl polymer is >50% by weight composed of polymers having a molecular weight of <100 000 daltons.

2. Active ingredient-containing polymer particles according to claim 1, which particles are in the form of an aqueous dispersion having a water content of 20-99.9% by weight.

3. Active ingredient-containing polymer particles according to claim 1, wherein the vinyl polymer consists of
A) 20-90% by weight alkyl esters of acrylic and/or methacrylic acid,
B) 80-10% by weight monomers having carboxyl groups and/or amino groups,
C) 0-40% by weight further monomers copolymerizable with A) and B).

4. Active ingredient-containing polymer particles according to claim 1, wherein the vinyl polymer component is >90% by weight composed of polymers having a molecular weight of <20 000 daltons.

5. Active ingredient-containing polymer particles according to claim 1, wherein liquid or oily substances having a solubility of <10 g/l of water are employed as active ingredients.

6. A method for administering active ingredients, which comprises swelling an aqueous polymer dispersion comprising a vinyl polymer which is composed of 10-80% by weight monomers comprising amino and/or carboxyl groups and which vinyl polymer is insoluble in one part of the pH range 0-10 and is soluble in another part of the range and which vinyl polymer is >50% by weight composed of polymers having a molecular weight of <100 000 daltons, in a pH range in which the vinyl polymer is insoluble, with 3-1000 parts of active ingredient per part of the vinyl polymer, with formation of active ingredient-containing polymer particles in a size range of 20 nm-8 μm, and administering the active ingredient-containing polymer particles.

7. Active ingredient-containing polymer particles according to claim 2, wherein the vinyl polymer consists of
A) 20-90% by weight alkyl esters of acrylic and/or methacrylic acid,
B) 80-10% by weight monomers having carboxyl groups and/or amino groups,
C) 0-40% by weight further monomers copolymerizable with A) and B).

8. Active ingredient-containing polymer particles according to claim 2, wherein the vinyl polymer is >90% by weight composed of polymers having a molecular weight of <20 000 daltons.

9. Active ingredient-containing polymer particles according to claim 3, wherein the vinyl polymer is >90% by weight composed of polymers having a molecular weight of <20 000 daltons.

10. Active ingredient-containing polymer particles according to claim 2, wherein liquid or oily substances having a solubility of <10 g/l of water are employed as active ingredients.

11. Active ingredient-containing polymer particles according to claim 3, wherein liquid or oily substances having a solubility of <10 g/l of water are employed as active ingredients.

12. Active ingredient-containing polymer particles according to claim 4, wherein liquid or oily substances having a solubility of <10 g/l of water are employed as active ingredients.
